(19) United States
(12) Patent Application Publication
(10) Pub. No.: US 2011/0112640 A1
(43) Pub. Date: May 12, 2011

(54) ADJUSTABLE LIGAMENT GRAFT FIXATION

(75) Inventors: Andrew Amis, Middlesex (GB); Thomas Boerger, Gibraltar (GI)

(73) Assignee: IMPERIAL INNOVATIONS LTD, London (GB)

(21) Appl. No.: 12/735,614

(22) PCT Filed: Jan. 28, 2009
(86) PCT No.: PCT/GB2009/000232

§ 371 Date: Nov. 12, 2010

(30) Foreign Application Priority Data
Feb. 2, 2008 (GB) 0801956.4

(51) Int. Cl. A61F 2/08

(52) U.S. Cl. 623/13.14

(57) ABSTRACT

A graft fixation device for fixing a ligament graft to a bone, the device comprising locating means for locating the device in the bone and graft support means arranged to support the graft, wherein the graft support means is adjustable to provide adjustment of the position of the graft relative to the bone.

### FIELD OF THE INVENTION

The present invention relates to ligament grafts, and in particular to the control of tension in ligament grafts. It has application in anterior cruciate ligament (ACL) grafts as well as grafts of other ligaments.

### BACKGROUND TO THE INVENTION

The need for a technique that allows post-fixation ligament graft re-tensioning after ACL, or other ligament, reconstruction is indicated by clinical observation of excessive anterior laxity in the immediate and early postoperative period in some people who undergo knee surgery. This may occur in cases where grafts are secured in both femur and tibia by interference screws. The causes may include viscoelastic graft stretching and fixation failure. In this context it is postulated that the graft loses its tendon characteristics and undergoes ‘ligamentization’. Mechanically this process is associated with a loss of initial tensile graft strength and increased susceptibility to creep. Soft tissue grafts may also slip past their fixation under cyclic loading. In addition, it is possible that there will be inadequate graft tension at the end of the surgical procedure due to technical reasons such as inadequate tibial reduction during graft fixation.

Re-tensioning procedures described in the art, other than thermal graft shrinkage, have historically always involved a revision of graft fixation; procedures such as removal and replacement of interference screws may damage the graft. Consequently, increased antero-posterior laxity is often accepted to avoid the sequelae of ACL revision.

ACL re-tensioning is theoretically feasible before graft incorporation into the bone tunnel has taken place. Little is known about the exact time it takes for secure soft tissue graft-tunnel osseointegration to occur. Reported timescales of 3 to 26 weeks have been based on animal models, but it is unclear whether this data represents the human situation. Time from surgery to graft osseointegration with interference screw fixation is believed to be in the region 6-15 weeks. Graft-femoral tunnel histology of knees with suspensory fixation or cross-pin fixation 9 to 11 weeks post-surgery showed a continuous soft-tissue envelope and only loose attachment between graft and bone [Nebelung et al., Arch. Orthop. Trauma Surg. 123: 158-163]. Hence, the time limit post-surgery for moving a tendon graft along a bone tunnel remains unclear.

### SUMMARY OF THE INVENTION

The present invention provides a graft fixation device for fixing a ligament graft to a bone, the device comprising a locating means for locating the device in the bone and a graft support means arranged to support the graft, wherein the graft support means is adjustable to provide adjustment of the position of the graft relative to the bone.

The graft support means may be arranged to be rotated to provide the adjustment. Alternatively it may be arranged to slide axially of the device, or to move radially of the device in the direction transverse to the device to provide the adjustment.

The graft support means may be formed integrally with the locating means so that both portions can be rotated together to provide the adjustment. In this case the graft support means and the locating means may comprise respective portions of the device. The graft support portion may, in this case, be adjusted by adjusting the position of the whole of the device. Alternatively the graft support means may be formed separately so that it moves relative to the locating means.

The device may further comprise locking means arranged to lock the graft support portion in any of at least two positions. The locking means may be arranged to lock the locating portion in position relative to the bone, which can lock the graft support portion if the two portions are fixed or locked relative to each other.

The locating means may be of substantially circular cross section having a central axis about which it can be rotated, and the graft support portion may be rotationally asymmetric about the axis so that its movement about the axis produces the adjustment. For example the graft support portion may be offset from the axis, to form a crank. Alternatively the graft support portion may have a rotationally asymmetric shape, for example triangular or elongate in cross section, so that it acts as a cam which provides the adjustment when rotated.

The device may further comprise a further locating means, the graft support means being located, at least partly, between the two locating means whereby the locating means are arranged to locate the device in the bone on opposite sides of a tunnel in the bone.

The present invention further provides a method of fixing a ligament graft to a bone, the method comprising providing a graft fixing having a locating means and a graft support means, locating the locating means in the bone, supporting the ligament graft on the graft support means, and adjusting the support means thereby to adjust the position of the graft relative to the bone.

The method may further comprise fixing the ligament graft, typically the opposite end of the graft, to another bone, and the adjustment be performed after that fixing has been performed so that the graft tension is adjusted after it has been fixed at both ends.

Embodiments of the invention can provide a novel means for post-fixation graft re-tensioning that is minimally invasive, easily accomplished and strictly extra-articular. Graft strain or damage at the anchor interface may be minimized.

Double looped semitendinosus and gracilis ACL grafts in conjunction with femoral cross pin fixation offer good biomechanical and clinical performance [Clark et al., Arthroscopy 14:258-267 and Kousa et al., Am. J. Sports Med. 31: 174-181]. Some embodiments of this invention are based on these established principles, but some feature a novel crank mechanism. In such embodiments the graft loop rests directly on the crank. Crankshaft rotation raises or lowers the loop within the femoral tunnel prior to the graft adhering permanently to the bone. At the time of graft fixation the cross-pin crank mechanism is positioned distally in the femoral tunnel. If graft post-operative re-tensioning is required, rotation of the crankshaft pulls the proximal end of the graft to a maximum at the opposing crank “crank up” position leading to significant reduction of excessive anterior-posterior (AP) laxity following ACL reconstruction. It has been shown previously that pulling the graft through the tunnel by adjusting its fixation increases graft tension and decreases AP knee laxity [Amis, J. Bone Joint Surg. Br. 71: 819-824].

Preferred embodiments of the invention can address the need for a technique that allows graft re-tensioning without undoing the femoral or tibial fixation, i.e. avoiding a revision procedure. The intervention of re-tensioning they provide can be simple, extra-articular and have minimal or no effect on graft integrity.

Preferred embodiments of the present invention will now be described by way of example only with reference to the accompanying drawings.

### BRIEF DESCRIPTION OF THE DRAWINGS

FIG. 1 is a side view of a graft fixation device according to an embodiment of the invention;

FIG. 2 is an and view of the device of FIG. 1;

FIG. 3 is an end view of a modification to the device of FIG. 1;

FIG. 4 is a detailed partial side view of the graft fixation device of FIG. 1;

FIG. 5 is a partial side view of a graft fixation device according to a further embodiment of the invention;

FIG. 6a is partially cut-away front view of a knee joint showing the device of FIG. 1 in operation;

FIG. 6b is a scrap section of the joint of FIG. 6a after adjustment of the device;

FIG. 7a is partially cut-away side view of the knee joint of FIG. 6a showing the device of FIG. 1 in operation;

FIG. 7b is a scrap section of the joint of FIG. 7a after adjustment of the device;

FIG. 8 is a bar chart showing the effect on graft tension of the device of FIG. 1;

FIG. 9 is a bar chart showing the effects on knee laxity of the device of FIG. 1 in use;

FIG. 10 is a section through a graft fixation device according to a further embodiment of the invention;

FIG. 11 is a cross section through a graft fixation device according to a further embodiment of the invention,

FIG. 12 is a cross section through a graft fixation device according to a further embodiment of the invention;

FIG. 13 is a front view of a graft fixing device according to a further embodiment of the invention;

FIG. 14 is a side view of the device of FIG. 13; and

FIG. 15 is a side view of a fixation pin according to a further embodiment of the invention.

### DESCRIPTION OF THE PREFERRED EMBODIMENTS

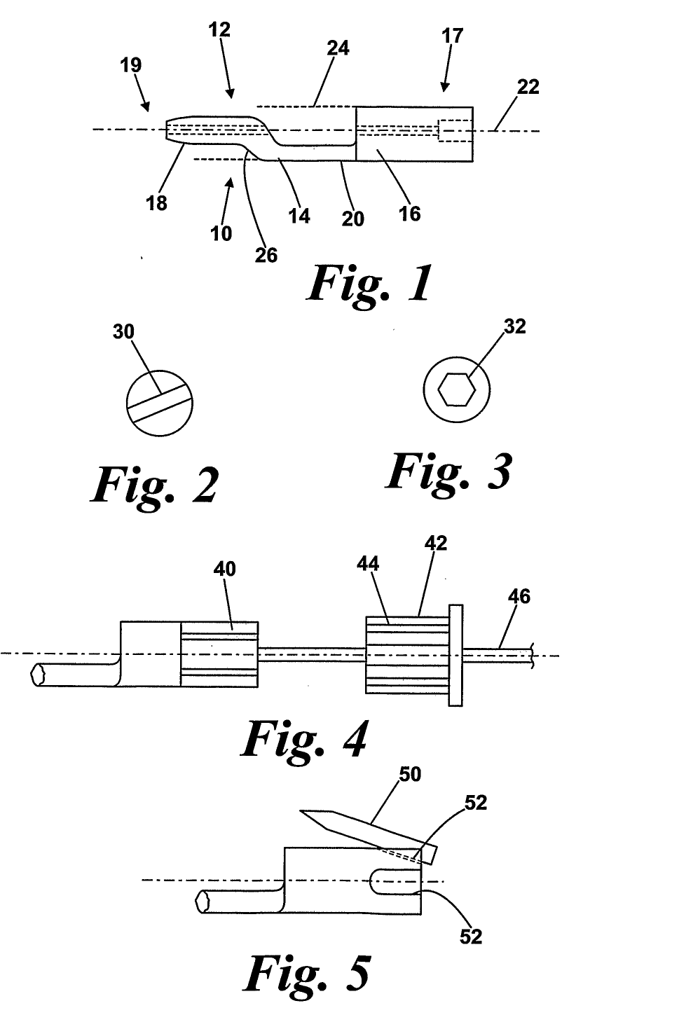

Referring to FIG. 1 an adjustable ACL fixation device takes the form of a cross-pin femoral hamstring graft anchor 10. The working end 12 of the anchor comprises a crankshaft 14 comprising a first locating portion 16 towards the proximal end 17, a second locating portion 18 at the distal end 19, and a centrally located graft support portion or crank 20 between the two locating portions 16, 18. The distal end 19 is tapered to ease passage through the end loop of a four-strand hamstring tendon ACL graft. The proximal locating portion 16 is of circular cross section and of a larger diameter than the distal locating portion 18, which is also of circular cross section. The two locating portions 16, 18 are both centered on a common longitudinal axis 22. The graft support portion 20 extends longitudinally from the distal end of the proximal locating portion 16. It is also of approximately circular cross section and of much smaller cross section than the proximal locating portion 16. It is offset from the longitudinal axis 22, but contained completely within the cylindrical envelope 24 defined by extending the cylindrical sides of the proximal locating portion 16. It is joined to the distal locating portion by cranked portion 26. The distal locating portion 18 and the cranked portion 26 are also located within this envelope. This ensures that the device can be inserted through a drilling designed to fit the proximal locating portion 16.

Referring to FIG. 2, a driving formation in the form of a simple transverse slot 30 is formed in the proximal end 17 of the pin 10 to enable it to be rotated about its longitudinal axis 22 by a standard screw driver. Any suitable drive formation, such as a hexagonal recess 32 as shown in FIG. 3, can be used.

Referring to FIG. 4, the proximal end of the proximal locating portion 16 is provided with longitudinal fluting 40. A cylindrical sheath 42 with matching longitudinal fluting 44 is arranged to fit over the end of the proximal locating portion with the fluting interlocking to prevent rotation of the sleeve relative to the anchor pin 10. The anchor pin 10 can be attached to a handle 46. The anchor pin 10 and handle are cannulated. The sheath 30 can be slid from the handle onto the proximal end of the anchor pin 10 to effect rotational locking by cortical engagement.

Referring to FIG. 5, in an alternative embodiment the locking mechanism comprises a simple pin 50 that can be located in any of a number of recesses 52 in the proximal end of the device and driven into the bone to lock the device.

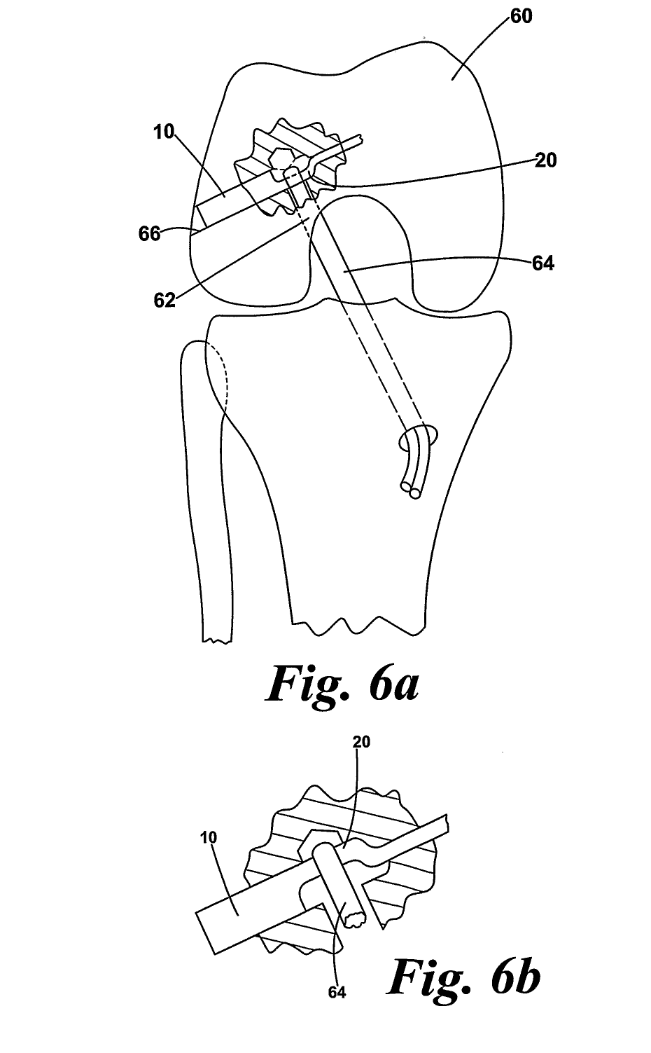

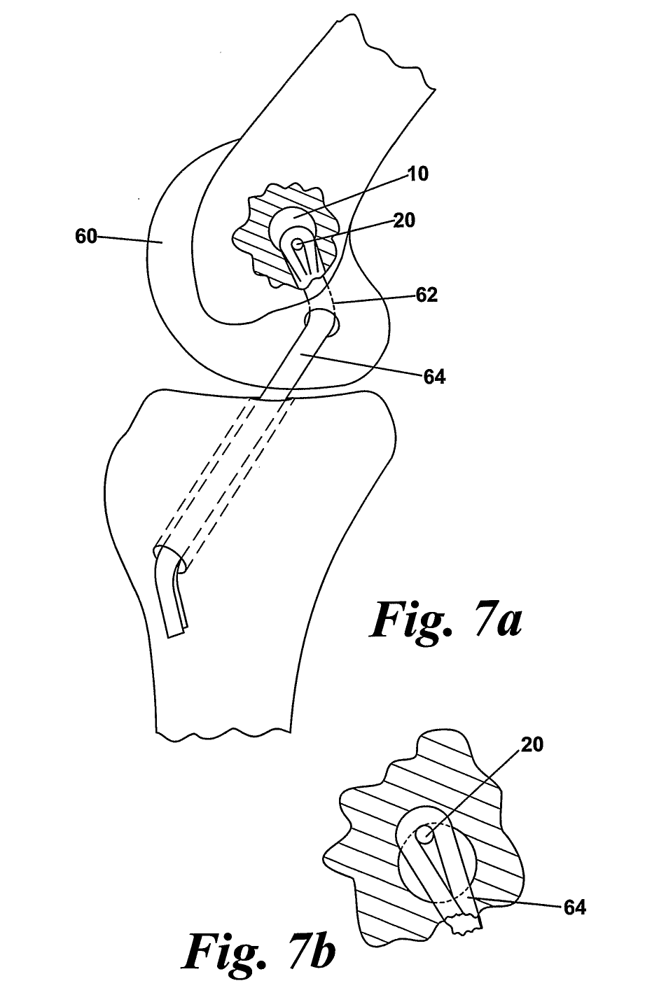

Referring to FIGS. 6a and 7a, when used to secure an ACL graft, the fixing pin 10 is embedded within the distal femur 60, transverse to the tunnel 62 in which the graft 64 is to be secured. In order to secure the graft 64 over the pin, the femoral graft tunnel 62 into which the ligature is to be secured is drilled in the femur, and a further transverse tunnel 66, which opens into the graft tunnel 62 is then drilled. A tunnel hook is then inserted up the graft tunnel 62 and a flexible wire is then passed through the transverse tunnel 66 and through the graft hook. The graft hook is pulled out of the graft tunnel 62 pulling a loop of the flexible wire with it. The graft is then looped through the flexible wire which is tensioned to pull the graft up the graft tunnel 62. The pin 10 is then slid along the flexible wire through the transverse tunnel 66 and the graft loop, so that the graft supporting portion 20 extends across the graft tunnel 62 and the locating portions 16, 18 are located and supported in the femur bone 60 on either side of the graft tunnel 62. The bottom end of the graft is then attached to the tibia, after having been passed through a tunnel in the tibia, to provide an initial tensioning of the graft.

FIGS. 6a and 7a show the anchor pin 10 in the down position, which is how it is first inserted. If the graft needs to be tightened, the pin 10 can then be rotated so that the graft supporting portion 20 moves upwards, hence pulling the graft along the graft tunnel 62 up into the femur, and locked in place. FIGS. 6b and 7b show the pin 10 with the graft 64 looped over it, with the crank 20 locked in the up position, after the graft has been tightened by adjustment of the device. Intermediate locking positions, between the two extreme up and down positions, are selectable to achieve the desired degree of ligament tensioning.

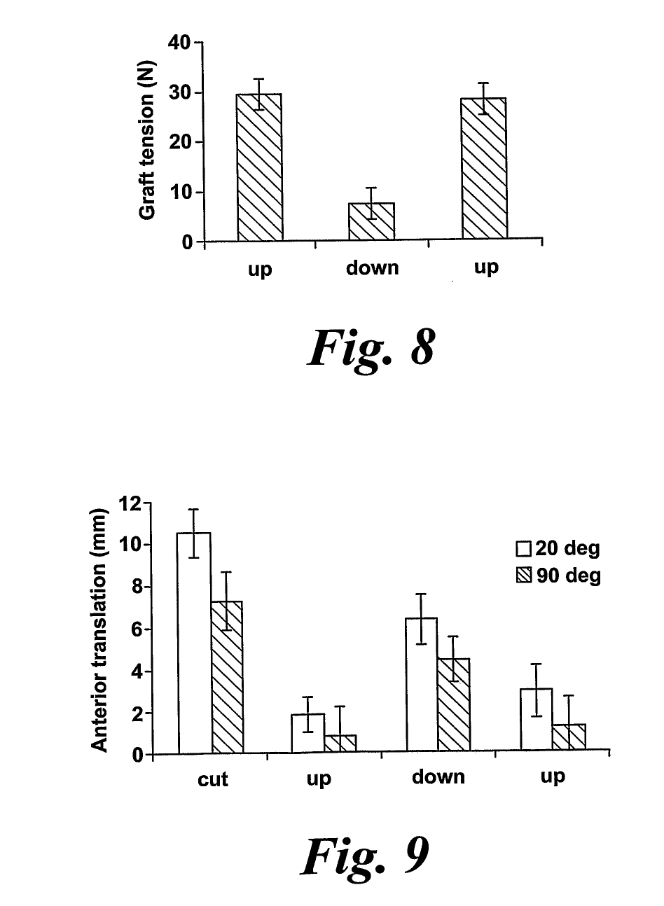

FIG. 8 shows changes in ACL graft laxity achieved using the anchor of FIG. 1 with the knee at 90 degrees of flexion. The three bars from left to right show the tension of the ligament: as reconstructed with the crank up; after lowering the crank; then after raising the crank again. Raising the crank increased graft tension significantly (p=0.0001). The values shown are mean values with standard deviation shown by the error bars, for a sample size of n=5.

FIG. 9 shows changes in AP laxity achieved using the anchor of FIG. 1. Average laxity of knees with intact ACLs is taken as baseline. The four pairs of bars from left to right show laxity as AP translation of the femur relative to the tibia: with the ACL cut, then after reconstruction, initially with the crank up, then down, and then up again. Raising the crank reduced AP laxity significantly at both 20 deg (p=0.009) and 90 deg knee flexion (p<0.0001). (White columns: 20 deg; grey columns: 90 deg). The values shown are mean values with standard deviation shown by the error bars, for a sample size of n=5.)

### EXAMPLE

Frozen cadaver knees with a mean age of 68.5 years, range 56-81, were used. Specimens were approximately 15 cm long above and below the joint line, and were sealed in polyethylene bags and stored at −20° C. After thawing the knees were dissected preserving only bone, joint, menisci, joint capsule and ligaments. The cut ends of the tibia and femur were cast into steel tubes using polymethylmethacrylate bone cement to provide stable fixation while testing.

Fresh frozen bovine extensor tendons were used as ACL grafts. These were harvested and trimmed into 5×200 mm long strips. Bovine extensor tendon grafts were used because their biomechanical properties are similar to human hamstring grafts. The reconstruction protocol used, including tunnel placement, tunnel orientation, magnitude of pretension force and degree of flexion at fixation was based on the ESSKA 1996 consensus workshop [Amis, Knee Surg. Sports Traumatol. Arthrosc. 6 Suppl 1:S2-12]. Other aspects of this work, such as the effects of freezing osseo-ligamentous specimens and laxity testing in a materials testing machine, are well-accepted in the literature [Arnoczky et al., J. Bone Jt. Surg. 64 A: 217-224]. Doubled upon itself each graft passed through a 10 mm diameter sizer. Whipstitches were applied to each end and the grafts were pre-conditioned by applying a tensioning force of 50N for 30 minutes.

After clearing ACL remnants a 10 mm/60° tibial tunnel was drilled exiting in the centre of the tibial attachment, using a 60° tibial drill guide (DePuy®, Johnson & Johnson®, Leeds, England). With the knee at 90° flexion, a 10×35 mm femoral tunnel was created at the 10:30 or 1:30 position respectively using a 5 mm transtibial offset femoral drill guide (Acufex®, Smith & Nephew Inc®, MA, USA). Using the Arthrex 90° c-arm and Transfix® instruments (Arthrex Inc., Naples, Fla., USA), a guidewire was placed through the femoral condyles, perpendicular to the femoral tunnel and through the tunnel hook fenestration. Over-drilling created a transverse 8 or 10 mm tunnel through the lateral femoral condyle which opened into the femoral tunnel. After exchanging the guidewire for a flexible wire the tunnel hook was withdrawn from the femoral tunnel, delivering a loop of flexible wire trans-tibially. The graft was doubled over the wire loop and pulled up into the femoral tunnel by opposing tension on the flexible wire ends.

The adjustable ACL fixation device was inserted by sliding it over the taut flexible guide wire through the graft loop and beyond the femoral tunnel into cancellous bone. Complete insertion was achieved once the thick proximal part of the crankshaft abutted against the graft loop. With the distal graft end held under tension alternating rotation of the crankshaft produced detectable shortening and elongation of the graft and ensured that the graft loop had settled in its correct position on the crank. Knees were reconstructed with a femoral anchor 10 mm in diameter with 8 mm between opposing crank positions.

The knees were preconditioned with 20 flexion-extension cycles. The distal graft ends were cemented into a metal tube which itself was linked via a load cell assembly to an external tibial post. With this set-up graft pre-tension could be adjusted. Knees underwent preconditioning and graft pre-tensioning with 40 N at 20° flexion for 30 minutes.

Antero-posterior laxity was tested using an Instron 1122 materials testing machine, (Instron Co., High Wycombe, UK). The femur was mounted in a stationary fixture on the base of the test machine angled to give either 20° or 90° knee flexion. The tibia was mounted horizontally in a fixture attached to the loadcell of the test machine, that was on the moveable crosshead. Thus, moving the crosshead upwards caused an anterior tibial translation (draw), representing either the Lachmann or anterior draw tests. The tibial fixture included linear bearings to allow secondary medial-lateral and proximal-distal translation movements, plus rotary bearings for secondary internal-external and varus-valgus rotations.

Prior to taking any measurements in each testing setup, the knee was subjected to 15 cycles of AP tibial displacement, between load limits of +/−150N, in order to precondition the soft tissues and minimize creep effects. After this, the crosshead continued to cycle between the same load limits at a speed of 50 mm/min while a force versus displacement graph was recorded.

The knees were tested in the ACL intact, ACL deficient and ACL reconstructed states at 20° and 90° flexion. In the reconstructed state testing was commenced in the crank up position (as tensioned initially) followed by testing in the crank down position (i.e. graft slackened) and finally in the crank up position (graft re-tensioned).

The graft tension was measured with no AP force imposed in the crank up position, in the crank down position, and again after return to the crank up position.

Changes in AP laxity were tested for statistical significance using a one-way Student's paired t-test, with p=0.05. The AP laxity of the reconstructed knees was compared to that of the intact or ACL-cut knees using a 2-way paired t-test, with p=0.017 for 95% alpha level applying a Bonnferroni correction.

The AP laxity results for the different stages of the procedure, are shown in FIG. 4. At 20° knee flexion, the AP laxity was a mean of 5.6 mm intact and increased to 16.2 mm after the ACL was cut. The ACL reconstruction reduced the laxity to 7.5 mm with the crank up initially. Rotating the crank down slackened the knee, leading to a mean AP laxity of 12.0 min; this was reduced 3.5 mm when the crank was rotated up, to leave 8.5 mm mean AP laxity. This was a significant (p=0.009) reduction in laxity. A similar pattern occurred at 90° knee flexion, again with a significant reduction in AP laxity when the crank was raised (p<0.0001). The mean AP laxity increased by 0.7 mm between the two sets of tests with the crank up.

The tightening and slackening of the knee, as the crank rotated, led to significant (p≦0.0001) changes in graft tension (FIG. 5).

The results were highly predictable. The adjustable ACL fixation device effected a graft shortening of 5 or 8 mm. With a graft orientation of about 60 degrees to the tibial plateau, the change in anterior displacement can be calculated applying 5 mm×cosine 60°=2.5 mm and 8 mm×cosine 60°=4 mm. As predicted the mean difference in laxity between up and down positions using an 8 mm crank, for both tightening and slackening procedures, was 4.0 mm at 20° knee flexion and 3.4 mm at 90° flexion.

The device allowed significant reduction in anterior laxity without undoing the tibial interference fixation. The alteration in anterior laxity was achieved simply by accessing the proximal end of the device and turning it by 180 degrees. Re-tensioning did not in any way require “intra-articular” access or direct interference with the graft itself.

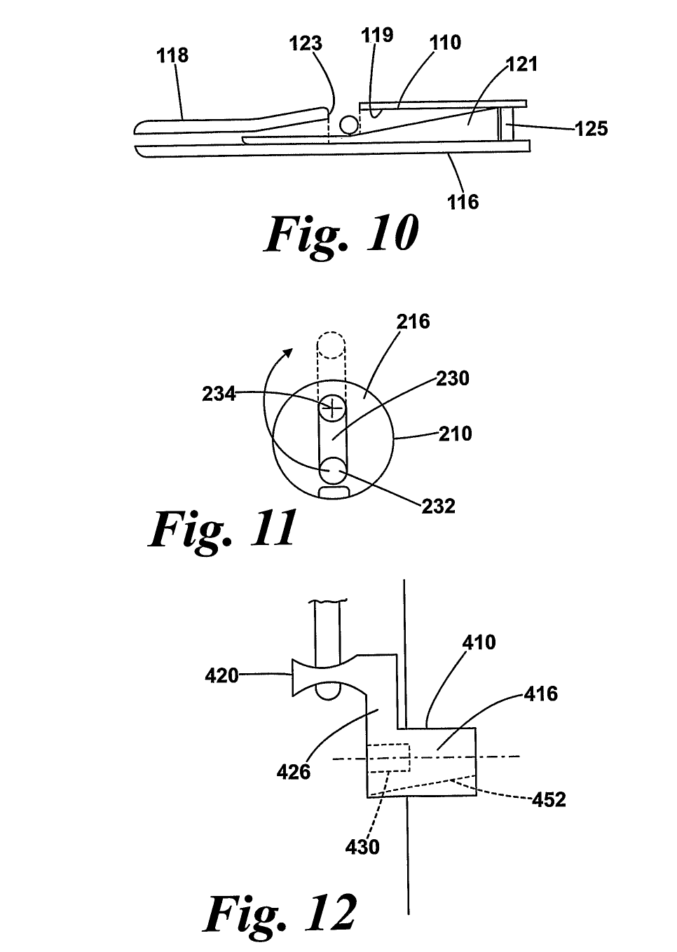

Referring to FIG. 10, in a further embodiment of the invention a cross pin ligament graft fixation device comprises a main pin body 110 having distal locating portion 118 and a proximal locating portion 116. The pin body 110 has a longitudinal passageway 119 extending through it, with a sliding adjustment member 121 located in it. The adjustment member is tapered, with a narrow distal end and a wider proximal end. The body 110 has an opening 123 in the centre between the two locating portions 116, 118 so that a ligament graft can be passed over the device and rest on the adjustment member 121. An adjustment grub screw 125 is arranged to push the adjustment member along the body 110 to raise the ligament graft with a wedge-type action.

Referring to FIG. 11, a further embodiment of the invention comprises a pin 210 shaped in a similar manner to the pin 10 of FIG. 1. However, instead of the whole pin being rotated, the device further comprises a separate crank member 230 which is rotatably supported near the top of the distal end of the proximal locating portion 216, and includes a graft support portion 232 offset from the axis of rotation 234. In the lowered position, as shown in solid lines in FIG. 11, the graft support portion 232 is located near the bottom of the proximal locating portion 216, but in the raised position, as shown in broken lines, it can be raised above the top of the proximal locating portion. This provides a greater level of adjustment than the device of FIG. 1.

Referring to FIG. 12, a further embodiment is a pin arranged for fixing a ligament graft to the side of a bone, for example for fixing the lower end of an ACL graft to the side of the tibia. The pin 410 comprises a locating portion 416 of circular cross section arranged to be inserted into a drilling in the side of the bone, and a graft support portion 420 in the form of a bar which is offset from the central axis of the locating portion 416 and joined to it by a cranked portion 426. A drive formation 430 is provided in the outer end of the locating portion 416. A recess 452 is provided in the side of the locating portion 416 so that it can be locked rotationally by a pin.

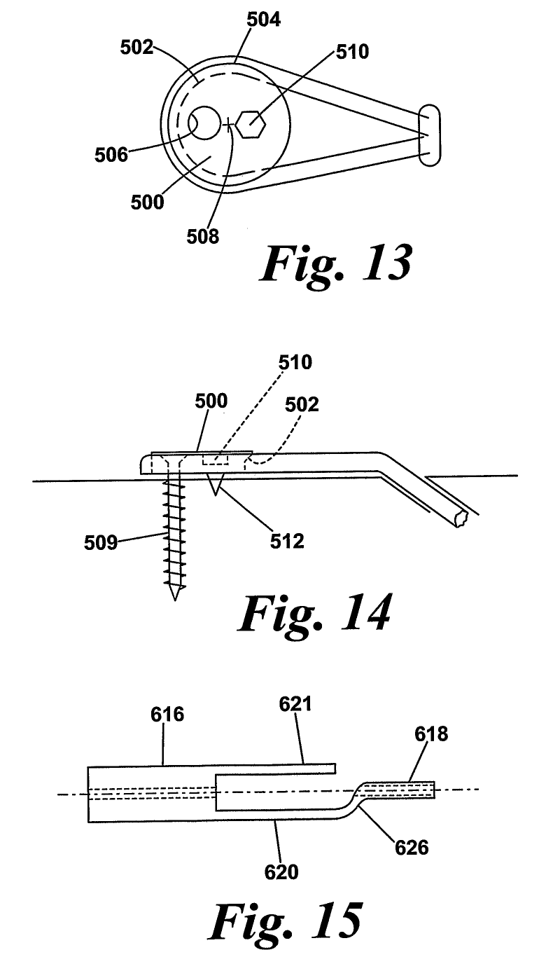

Referring to FIGS. 13 and 14 a fixation device according to a further embodiment of the invention comprises a flat circular cam member 500 with a circumferential groove 502 around its outer edge 504. A mounting hole 506 is provided through the cam member 500 in an eccentric position offset from its centre 508, through which a locating device such as a standard bone screw 509 can be introduced. The screw 509 can then be secured in the bone to provide a rotational axis about which the cam member 500 can rotate. A drive formation is formed in the top of the cam member 500 in the form of a hexagonal recess 510 arranged to receive an Allen key. A small locking spike 512 projects from the underside of the cam member 500 and is arranged to project into the bone to lock the cam member 500 in rotation.

In operation the device of FIGS. 13 and 14 is used in a similar way to that of FIGS. 11 and 12 with the cam member 500 being locked and released in rotation by tightening or releasing the screw 509 to drive the spike 512 into the bone or allow it to rise out of the bone. This device can be used with a standard pin at the top of the ACL, so that it provides the only means of adjustment. Alternatively it could be used in conjunction with an adjustable pin such as that of FIG. 1 to provide additional adjustment.

Referring to FIG. 15, a graft fixation pin is arranged to be used in a similar manner to that of FIG. 1. It includes all of the features of the pin of FIG. 1 indicated by the same reference numerals increased by 600. However, a winding projection 621 projects axially from the distal end of the locating portion 616 in a position diametrically opposite the graft support portion 620. The winding projection 621 extends axially about the same distance as the graft support portion 620 but its distal end is free and unsupported and spaced from the cranked portion 626.

In use the graft support portion 620 can again be inserted through the loop at the end of the graft, and rotation of the pin will pull the graft upwards. However the pin can be rotated through more than 180° and in fact through any number of turns and the graft will be wound round the graft support portion 620 and the winding projection 621 to continuously increase the tension in the graft.

1. A graft fixation device for fixing a ligament graft to a bone, the device comprising locating means for locating the device in the bone and graft support means arranged to support the graft, wherein the graft support means is adjustable to provide adjustment of the position of the graft relative to the bone.

2. A device according to claim 1 wherein the graft support means is arranged to be rotated about an axis to provide the adjustment.

3. A device according to claim 2 wherein the graft support means is formed integrally with the locating means so that both can be rotated together to provide the adjustment.

4. A device according to claim 2 wherein the locating means is of substantially circular cross section having a central axis about which it can be rotated.

5. A device according to claim 4 wherein the graft support means fits within a cylindrical volume of the same diameter as the locating means so that the graft support means can be inserted through an aperture in which the locating means can fit to locate the device.

6. A device according to claim 2 wherein the graft support portion is rotationally asymmetric about the axis so that its movement about the axis produces the adjustment.

7. A device according to claim 6 wherein the graft support portion is offset from the axis.

8. A device according to claim 6 wherein the graft support means is eccentric relative to the axis.

9. A device according to claim 2 further comprising winding means arranged to be rotated to wind the graft around the graft support means.

10. A device according to claim 1 further comprising locking means arranged to lock the graft support means in any of at least two positions.

11. A device according to claim 10 wherein the locking means is arranged to lock the locating means in position relative to the bone.

12. A device according to claim 1 further comprising a further locating portion, the graft support portion being located, at least partly, between the two locating portions whereby the locating portions are arranged to locate the device in the bone on opposite sides of a tunnel in the bone, and the graft support portion is arranged to support the graft in the tunnel.

13. A device according to claim 1 wherein the graft support portion is movable relative to the locating portion to provide the adjustment.

14. A device according to claim 1 in the form of a fixation pin for insertion into a bore in the bone.

15. A device according to claim 1 wherein the locating means has one end arranged to be inserted into the bone and one end arranged to support the graft support means, and the graft support means is arranged to lie against the surface of the bone.

16. A method of fixing a ligament graft to a bone, the method comprising providing a fixing pin having a locating portion and a graft support portion,
locating the locating portion in the bone, supporting the ligament graft on the graft support portion, and adjusting the support portion thereby to adjust the position of the graft relative to the bone.

17. A method according to claim 16 further comprising locking the support portion in its adjusted position.

18. A fixation pin device substantially as hereinbefore described with reference to any one or more of the accompanying drawings.
